(19) United States
(12) Patent Application Publication
(10) Pub. No.: US 2023/0355168 A1
(43) Pub. Date: Nov. 9, 2023

(54) METHOD AND DEVICE FOR DETECTING AND THERAPEUTICALLY MONITORING KNEE LIGAMENT INJURIES

(71) Applicant: GENOUROB, Laval (FR)

(72) Inventors: Stéphane NOUVEAU, L'Huisserie (FR); Henri ROBERT, Laval (FR)

(21) Appl. No.: 18/026,446

(22) PCT Filed: Sep. 15, 2021
(86) PCT No.: PCT/FR2021/051585

§ 371 Date: Mar. 15, 2023

(30) Foreign Application Priority Data
Sep. 15, 2020 (FR) 2009339

(51) Int. Cl. A61B 5/00; A61B 5/107; A61B 5/11

(52) U.S. Cl. CPC A61B 5/4528; A61B 5/1071; A61B 5/1121; A61B 5/6828; A61B 5/704

(57) ABSTRACT

According to the method of the invention, a translational movement of the tibia of a patient is caused by a first motor (9b) for moving a thrust member (9a) of the patient's calf, and is measured by a first sensor (15a) located on the Tibial Tuberosity, Anterior (TTA). The tibia is caused to rotate by a second motor (12) for rotating a foot support (11b) of the patient, and is measured by a second sensor (14b) located as close as possible to the TTA. The respective operations of the first motor (9b) and of the second motor (12) are correlated by a control member, by being mutually controlled according to pre-set and reproducible force models.

### FIELD OF THE INVENTION

The invention relates to the field of methods and devices for measuring, characterizing and monitoring injuries to the knee ligaments of a patient. More specifically, the invention relates to a method and a device for implementing same, making it possible to measure a rotational instability of the knee in combination with a measurement of a translational movement of the tibia relative to the femur of a patient, at different degrees of bending of the knee.

### TERMINOLOGY

In the present patent, the following abbreviations, usual for the person skilled in the art, will be used:
ACL for “anterior cruciate ligament”
TTA for “Tibial Tuberosity, Anterior”

### PRIOR ART

Among the measurement devices used to establish a diagnosis, devices that make it possible to measure and follow an anterior cruciate ligament injury of a patient's knee are known. The anterior cruciate ligament (ACL) is composed of numerous collagen fibers that can be separated into two bundles respectively inserted into the tibia and the femur, namely an anteromedial bundle and a posterolateral bundle. Depending on the trauma suffered by the patient, rupture of the ACL may be total or partial.

It is essential to be able to troubleshoot and quickly develop a precise diagnosis of an injury to the ACL, especially in the case of partial injury which is difficult to detect on the fly after physical exertion by the patient. Such early diagnosis is especially crucial for an athlete, whose performance is affected by knee solidity. Diagnosing the start of an ACL tear as soon as possible makes it possible to leave time for the patient to heal, avoiding a risk of permanent ACL rupture, and to set up a course of rehabilitation suitable for protecting the ligament during its healing.

It is important that the diagnosis be established as accurately as possible in order to optimize the patient's course of rehabilitation in the context of therapeutic monitoring. The patient's state must be monitored through repeated, precise, and reliable measurement of the progress of an improvement in the patient's ACL, which will help to optimize their therapeutic monitoring.

For this purpose, devices for detecting a partial injury to the knee's ACL are known, such as for example the device described by document EP2699160-B1 (GENOUROB). According to this document, the device comprises a seat for the patient to sit on, including a support platform for supporting the patient's leg which is pivotably mounted on the squab of the seat. The patient's knee is immobilized on the platform and the patient's foot is immobilized on a foot support rotatably mounted on the platform.

The platform is equipped with a thrust member to push the patient's calf, which is mounted so as to be translationally movable on the platform. A first sensor measures a translational movement of the anterosuperior face of the patient's tibia as a result of the push exerted on the calf by said thrust member. The foot support is equipped with a second sensor arranged as an inclinometer, to identify an angle of rotation of the tibia as a result of the foot support being rotated.

From the measurements carried out by the first sensor and the second sensor, an analysis logic system identifies a possible partial injury to the anteromedial ligament and/or the posterolateral ligament. To do this, the measurement is then established on the basis of a comparison between measurements performed successively on the one hand on the leg potentially affected by an ACL injury and on the other hand the patient's healthy leg. Measuring the internal and/or external rotation of the foot of a tested leg makes it possible to detect any potential rotational instability of the knee during a translational movement of the tibia relative to the femur caused via the thrust member, and this at different degrees of bending of the knee. This makes it possible to bring to light a partial injury to one of the two bundles of the ACL of the knee and rotational instability of the knee. Such a technique is therefore advantageous in that it makes it possible to accurately detect an injury to the anteromedial bundle and/or an injury to the posterolateral bundle that take into account the rotational instability of the knee.

Japanese patent application JPH09276252A proposing a knee support and an ankle support, and a seat with a backrest mounted on the upper end of the base is also known. It comprises a forward traction mechanism, a rearward pressure mechanism and a measurement means are arranged on the base of the knee support. The calf of a subject is pushed forward to impart a forward traction force to the knee joint ligament, and the upper surface of the ball joint located on the hamstring support is pressed by the mechanism imparting rearward pressure in order to impart a rearward pressure force onto the ball joint.

The forward movement of the vertically moved tibia is measured as a function of the movement of the upper surface of the ball joint by the measurement means.

Patent WO2008/040790 is also known, describing a device for detecting and monitoring an injury (rupture or tear) to the ACL by anterior translation of the tibia relative to the femur comprising a support of the lower limb characterized in that it comprises a means for pushing the posterosuperior face of the calf and a movement sensor of the tibia that is positioned on the anterosuperior face of the Tibia, on the TTA (Tibial Tuberosity, Anterior).

Patent EP2839775 proposes a device for determining a relative position of a femur relative to a tibia comprising at least:
a first measuring member intended to be positioned against the tibia and comprising means for generating a signal representative of a position of the measuring member;
a second and a third measuring member intended to be positioned relative to the femur by each encircling the thigh associated with the femur at different heights, the second and the third measuring member each including means for generating a signal representative of a position of the associated measuring member;
a computing member including communication means for receiving at least the signals generated by the first, second and third measuring members, comparison means that compare the signals of the second and third members to deduce therefrom an absolute position of the femur, and processing means that analyze the signal of the first measuring member and the absolute position of the femur to deduce therefrom a relative position of the femur relative to the tibia.

Patent EP3178381 has as its subject matter an evaluation device for evaluating morphological parameters of a lower limb of a patient, especially during a preoperative or postoperative consultation, the evaluation device being characterized in that it comprises at least:
a femoral set of inertial sensors comprising three femoral gyrometers and three femoral accelerometers, the femoral gyrometers being configured to respectively generate signals representative of their respective angular velocities around the three dimensions of a Galilean reference frame, the femoral accelerometers being configured to generate signals representative of their respective linear accelerations according to the three dimensions of the Galilean reference frame,
a tibial set of inertial sensors comprising three tibial gyrometers and three tibial accelerometers, the tibial gyrometers being configured to respectively generate signals representative of their respective angular velocities around the three dimensions of a Galilean reference frame, the tibial accelerometers being configured to generate signals representative of their respective linear accelerations according to the three dimensions of the Galilean reference frame,
a femoral support configured to maintain the femoral assembly of inertial sensors on the patient's thigh, the femoral support having: i) a proximal femoral attachment portion configured to secure the femoral support to a proximal portion of the thigh, and ii) a femoral attachment distal portion configured to secure the femoral support to a distal portion of the thigh,
a tibial support configured to maintain the tibial assembly of inertial sensors on the patient's thigh, the tibial support having: i) a proximal tibial attachment portion configured to secure the tibial support to a proximal portion of the leg, and ii) a tibial attachment distal portion configured to secure the tibial support to a distal portion of the leg,
a malleolar probe attached to the tibial support, the malleolar sensor comprising a medial branch and a lateral branch which are articulated to the tibial support so as to be movable between:
   i) a contact configuration, wherein a contact area of the medial branch and a contact area of the lateral branch are brought into contact respectively with the medial malleolus and with the lateral malleolus; and
   ii) a remote configuration, wherein the medial branch and the lateral branch are respectively distant from the medial malleolus and lateral malleolus, and
   a computing unit configured to receive the signals generated by the femoral gyrometers, by the femoral accelerometers, by the tibial gyrometers and by the tibial accelerometers, the computing unit being further configured to:
      receive said generated signals when the tibial set of inertial sensors follows a first circular movement induced when the lower limb in extension or hyperextension rotates about the patient's femoral head,
      calculate, from the received signals, the position of the center of the femoral head,
      receive said generated signals when the femoral set of inertial sensors is immobile, the patient thigh being in stationary and in extension or hyperextension, and when the tibial set of inertial sensors follows a second circular movement induced by at least one leg bending greater than 60 degrees, for example, greater than 90 degrees,
      calculate, from the received signals, the position of the center of the knee,
      receive said generated signals when the medial branch and the lateral branch are brought into contact respectively with the medial malleolus and the lateral malleolus while the foot is immobile, and
         calculate, from said received signals, the position of the center of the ankle as being the middle of the segment delimited by the contact area of the medial branch and the contact area of the lateral branch when the medial branch and the lateral branch are in the contact configuration.

### OVERVIEW OF THE INVENTION

The subject of the invention is a method for evaluating an injury to the cruciate ligament of a patient's knee, by implementing a device of the aforementioned type according to which measurements are carried out in order to identify a possible rotational instability of the knee in combination with a translational movement of the tibia relative to the femur, at different degrees of bending of the knee.

The device is of the aforementioned type comprising a seat located on a base (6) including a squab extended by a backrest tiltable relative to the squab, and a platform for resting a patient's leg which is mounted so as to be pivotably movable relative to the squab. A thrust member to push the patient's calf is mounted so as to be translationally movable on the platform and a first sensor is assigned to the measurement of a translational movement of the patient's tibia as a result of the push exerted against the calf. A foot support is mounted so as to be rotationally movable at the end of the platform, in order to cause the tibia to be rotated and a second sensor is assigned to measuring an angle of rotation of the tibia. A logic system for analyzing measurements made by the first sensor and by the second sensor makes it possible to diagnose and/or monitor the progress of a possible partial injury to the ACL of the knee.

It has appeared in use that such a device and its methods of implementation could be improved to enhance its ergonomics, the quality of the diagnosis obtained and the adaptation of a therapeutic monitoring of the patient, including, where appropriate, in a post-operative phase.

On the basis of this observation, the aim of the invention is especially to propose such improvements to the devices of the prior art.

One related objective is to allow an injury to lateral capsular ligament structures to be detected at the knee joint such as especially the anterolateral ligament or other lateral capsular ligament structures—which have a strong influence on the rotational instability of the knee, thus making its objective assessment very difficult.

Another related objective of the invention is to make it possible to quickly detect an injury to the ACL of a patient's knee, while being able, if possible—especially during therapeutic monitoring—to ignore without prejudice the relevance of the diagnosis obtained to then compare measurements carried out for the leg affected by an injury to the ACL of the knee and for the patient's healthy leg.

Another related objective of the invention is to seek conditions for measuring a rotational instability of the knee in combination with a translational and rotational movement of the tibia relative to the femur, making it possible to increase the relevance of the diagnosis which is deduced therefrom and/or to avoid a loss of reliability of the measurements carried out in the event of translational or rotational tibial tests alone.

Another objective of the invention is to improve the ergonomics of the device, by allowing the practitioner to carry out in a fast and reliable manner various translational and rotational measurements of the tibia during therapeutic monitoring of the patient, from which they can establish a precise diagnosis of the evolution of the injury to the ACL of the patient's knee by easily adapting their examination conditions.

Another related objective is to obtain reliable and precise measurements promoting the relevance of their comparison with similar measurements subsequently carried out in the context of therapeutic monitoring, especially in a post-operative phase of the patient. More precisely, the aim is to take into consideration an evaluation of the progress of the resistance of a transplant during repeated tests throughout the rehabilitation and the resumption of a sport played by the patient, in order to detect a potential expansion of the transplant.

More precisely, it is aimed at making it possible to evaluate an apparent distension of the transplant from measurements carried out via the device during the first months, to correct exercises performed by the patient in the post-operative phase in order to make them less restrictive. It is thus ultimately sought to allow the transplant to be re-tightened by virtue of the process of reconstruction of the graft of the transplant, which is not ordinary healing, by promoting early and gradual post-operative care for the patient.

It is also more precisely targeted to provide precise analysis of the translation associated with the internal rotation of the tibia, especially to achieve an objective and precise study of potentially existing injuries to lateral, anterolateral and/or posterolateral capsular ligament structures, on the basis of a comparative analysis of the precise measurements carried out in a post-operative phase of the patient.

To do this, and according to one approach of the invention, both in its generality and in its specificities, the invention proposes via a control member to correlate a motorized rotation of the patient's foot and a motorized movement in translation of the tibia relative to the femur at different degrees of bending of the knee caused by the thrust member.

The rotation of the foot support controlled by the control member in correlation with the thrust exerted against the patient's calf, at different degrees of bending of the knee, has the advantage of giving rise to an equivalent to jump tests, which are problematic for a practitioner to carry out manually on an awake subject. This also improves the performed examination of the patient under optimum conditions of reproducibility during therapeutic monitoring of the patient.

Furthermore, such correlation makes it possible to easily produce such jump tests at different thresholds for stressing the tibia, according to various methods of inspection predefined and/or adaptable by the practitioner depending on the progress of the injury to the ACL during therapeutic monitoring of the patient.

It is thus not only possible to overcome the difficulty for a practitioner to carry out the test manually, but also to obtain, on the one hand, reliable and precise measurements and, on the other hand, a possibility of reproducing, in the same manner, in the context of therapeutic tracking, the combined movements of rotating the foot and of putting the tibia into translation relative to the femur under the control of the control member.

The practitioner is also able to adapt the correlated servo-control of the rotational and translational movements imparted to the tibia, by being able to modify the forces of stress on the tibia depending on the progress of the healing of the injury to the ACL and/or depending on the search by the practitioner of an identification of a specific ligament injury to the patient's knee.

The rotation of the foot support by the control member is preferably carried out at constant speed, according to a torque supplied by the motor driving the rotation of the foot, which is selected from force laws listed in a range of several torques to be applied between 3 N/m and 8 N/m (N/m: Newton per meter of leverage), as a function of an amplitude of rotation of the foot and/or of a motorized translational movement of the tibia via the thrust member.

The foot support can be rotated at an internal angle and then following an external angle of values varying for example over an angle range that may extend up to 45 degrees on either side of a neutral position of the foot in a position at the zenith of the anterior tibial tuberosity, depending on the torque applied. The motorized translational movement of the tibia is achieved by applying—according to an amplitude of movement of the thrust member—a force that can for example reach 134 N (N: Newton)—or according to the international reference 30 lbs (lbs: pounds)—, in accordance with listed force laws.

Such a servo-control makes it possible to measure either simultaneously or consecutively on the one hand a rotation angle of the tibia and, on the other hand, a translational movement of the tibia, while allowing logic correlation by a system for analyzing the measurements respectively obtained.

Especially, in the context of therapeutic monitoring, a loss of reliability is thus avoided in the combination of the measurements carried out relating to the mobility of the tibia in translation relative to the femur and to its rotation, with respect to such measurements obtained by movements of the tibia that are not servo-controlled and/or are necessarily carried out simultaneously in rotation and in translation.

Furthermore, the practitioner has the possibility of carrying out diversified measurements at different given degrees of bending of the knee, according to various selectable conditions for rotating the tibia combined with its translational movement, and this simultaneously and/or consecutively. The diversification of the measurements obtained allows the practitioner to compare the results of the logical analysis obtained to increase the relevance of their diagnosis.

It emerges that the precision, diversity and adaptation that can be configured by the practitioner of the means of capturing measurements, makes it possible to quickly and easily establish a reliable and relevant diagnosis from said correlation of the motorizations respectively assigned to the rotation of the tibia and to its translational movement, and this independently of simultaneous and/or successive implementations of said motorizations.

This is particularly useful in the context of early detection of a possible injury to the ACL of the knee. This is also particularly useful in the context of therapeutic monitoring of the patient, on the basis of a modelling of the mobility conditions of the tibia via said correlated motorizations on the basis of pre-set force models, allowing their subsequent reproduction either identically and/or adapted to the context of the therapeutic monitoring of a specific patient.

It is also proposed to place the second sensor for measuring a rotation of the tibia as close as possible to the anterior tibial tuberosity—hereinafter referred to as TTA. Such a position of the second sensor provides reliable and precise measurements of the angle of rotation of the tibia as close as possible to the knee joint, which improves the relevance of the diagnosis obtained, especially due to its distance from the foot to which the rotation of the tibia is applied.

This makes it possible to avoid interference on the measurements carried out by the second sensor and/or to obtain precise measurements of the actual rotation of the tibia by comparison with a given angle of rotation of the foot support. Potential interference on the measurements provided by the second sensor that can affect their relevances is also avoided. Such interferences are likely to occur in the event of an overestimation of the amplitude of the angle of rotation of the tibia as a result of the movements induced by the articulations of the foot and the ankle, and/or because of the mass of collagen tissue interfaced between the patient's foot and knee.

An assistance device is preferably used to guide the practitioner in the positioning of the first sensor and/or of the second sensor on the patient. A signal transmitter, such as sound and/or visual signals, can be used for this purpose on the basis of a prior calibration of the sensors in a reference position. Such a calibration is also advantageously used for the construction of force models used by the control member in order to reproduce and/or adapt—upon therapeutic monitoring of the patient—the conditions for stressing the tibia and consequently the measurements previously carried out and stored for a specific patient.

The second sensor can be located on the leg segment as close as possible to the anterosuperior part of the tibia, preferably on the tibial edge (close to the TTA). The second sensor is potentially located as a temporary substitute for the first sensor preferably located in the active position against the TTA. This is especially useful for optimally placing the second sensor in application close to the TTA without excluding a measurement prior to or subsequently carried out by the first sensor then located on the TTA, in the case of respective measurements of the rotation of the tibia and of its translational movement which are carried out alternately under control of the control member.

The potential presence of the first sensor for measuring the translational movement of the tibia, which is preferably located in application against the TTA, is therefore taken into account.

In the case of simultaneous measurements of the angle of rotation of the tibia and of its translational movement, the second sensor is then located as close as possible to the TTA in the immediate vicinity of the first sensor following the extension of the patient's leg, for example by being mounted on a legging that at least partially envelops the patient's calf. In the case of consecutive measurements of the angle of rotation of the tibia and of its translational movement, the second sensor can then be located on the TTA as a temporary substitute for the first sensor which are then advantageously jointly mounted movably on an articulated beam.

Furthermore, the seat is also equipped with various motor members via which the patient is installed on the seat in the examination position. Such motor members are tasked at least with the relative positioning of the components of the seat, especially with positioning in relative inclination between the squab and the backrest, with positioning in relative inclination between the platform and the squab, and additionally with positioning the squab in height.

It is therefore also proposed to model for a given patient the initial examination conditions of a patient, and therefore the conditions under which the mobility measurements of the tibia are made. Such a model of the patient's examination conditions is carried out on the basis of storing the relative positions between the movable components of the seat, from which the various motor members assigned to the seat components are stored. Thus, the conditions of an initial examination with different degrees of bending of the patient's knee can be reproduced accurately during their therapeutic monitoring, especially via the control member.

In light of what has just been explained, a method of the invention is a method for evaluating an injury to the ACL of a patient's knee, at least by measuring a rotational instability of the knee in combination with a measurement of a movement of translational movement of the tibia relative to the patient's femur at different degrees of bending of the knee.

The method implements a device at least comprising a seat in which to place the patient including a squab extended by a backrest tiltable with respect to the squab, and including a support platform for supporting a leg of the patient which is pivotably mounted in position on the squab. The platform is equipped with a first member for immobilizing the patient's knee on the platform, a foot support which is provided with a second member for immobilizing the patient's foot and which is mounted rotationally movable on the platform, and a thrust member of the patient's knee mounted translationally movable on the platform, the device being equipped with a first sensor for measuring a translational movement of the tibia relative to the femur located on the TTA, and a second sensor for measuring an angle of rotation of said tibia close to the TTA.

According to such a method, said assessment of an injury to the ACL is diagnosed from a logical collection and processing by a measurement analysis system comprising at least a first measurement provided by the first sensor and a second measurement provided by the second sensor for each of said different degrees of bending of the knee.

In this context, a method according to the invention has the following features:
measuring the translational movement of the tibia is carried out by the first measurement sensor from a translation of the thrust member on the platform which is performed by a first motor,
measuring the angle of rotation of the tibia is carried out by the second sensor located as close as possible to the TTA of the patient's knee, from a rotation of the foot support which is performed by a second motor,
The respective operations of the first motor and of the second motor are correlated by a first control module that a control member includes,
The respective activation conditions of the first motor and of the second motor are mutually controlled, especially with regard to their kinematics, their dynamics and their simultaneous and/or consecutive operation, according to at least one listed model of stressed forces for stressing the tibia of a specific patient which is pre-set and reproducible and/or adaptable according to the progress of the injury to the ACL of the patient's knee, especially in the context of therapeutic monitoring of the patient for which said at least one force model is listed,
said at least one force model combines, for each of the degrees of bending of the patient's knee, on the one hand a first force law relating to the translational movement of the tibia, via the thrust member moved in translation on the platform by the first motor, and on the other hand a second force law relating to the rotation of the patient's foot, via the foot support, by the second motor for rotating the foot support.

According to one implementation of the method, the second sensor is located near the thrust member following the extension of the leg, the first sensor being located on the TTA and the second sensor being located adjacent to the first sensor.

According to this case, the second sensor is for example mounted on a legging at least partially installed around the patient's calf.

According to one variant, the second sensor and the first sensor are mounted on an articulated beam which is attached to the platform, on which the second sensor and the first sensor are installed selectively separately and/or jointly individually on the beam.

In other words, only the first sensor can be installed on the beam and the second sensor can be located adjacent to the first sensor, for example being mounted on said legging. According to one variant, the first sensor and the second sensor can be jointly installed on a same said beam, or be installed on said respective beams. In this case, the beam(s) are equipped with respective mounting members of the first sensor and of the second sensor on the beam(s), in respective adjustable positions.

This makes it possible to selectively carry out measurements of mobility of the tibia on the one hand by respective applications on the TTA for the first sensor and in the vicinity of the TTA for the second sensor in order to carry out said measurements simultaneously, and/or by individual applications alternating with the first sensor or of the second sensor against the TTA, in order to carry out said measurements consecutively to improve if needed the precisions thereof.

Thus, the second sensor may be located on the TTA as a temporary substitute for the first sensor. The first sensor is for example mounted in isolation on the beam and the second sensor is mounted on the legging, and/or the first sensor and the second sensor are jointly mounted on a beam or are mounted on respective beams, by being selectively applied alternately against the TTA.

Thus, according to first conditions of examination, the measurement of the translational movement of the tibia by the first sensor and the measurement of the angle of rotation of the tibia by the second sensor, are carried out simultaneously under the control of the control member in accordance with said at least one force model.

Thus again, according to second conditions of examination, the measurement of the translational movement of the tibia by the first sensor and the measurement of the angle of rotation of the tibia by the second sensor, are carried out consecutively under the control of the control member in accordance with said at least one force model.

According to one implementation of the method and prior to an evaluation examination of an injury to the ACL of a specific patient's knee, the first sensor and the second sensor are calibrated at respective reference positions, by examining a healthy leg of the patient subjected to said at least one force model.

Such a calibration being carried out and taking into account the correlated operations of the first motor and of the second motor according to said at least one pre-set force model, the examination of the patient's knee during therapeutic monitoring may be carried out directly without any new measurements performed on the healthy leg.

According to one implementation of the method, the control member includes a second servo-control module correlated with at least one third motor for positioning in relative tilt between the squab and the backrest, a fourth motor for positioning in relative tilt between the platform and the squab, and a fifth motor for positioning in height the squab. The respective strokes of the third motor, the fourth motor and the fifth motor are especially mutually controlled according to at least one configuration model of the seat that is pre-set and reproducible for a specific patient. Said configuration model of the seat is especially constructed during an initial examination of the patient to detect a possible injury to the ACL.

Another subject of the invention is a device configured for implementing a method according to the invention. The device of the invention relates to the type of device comprising said seat including a squab extended by a backrest tiltable with respect to the squab, and comprising a support platform for supporting a patient's leg which is pivotably mounted in position on the squab and which is equipped with a first member for immobilizing the patient's knee on the platform, such as, for example, arranged as a knee-tightening strap on a support base of the knee.

The platform is also equipped with a thrust member for pushing the knee, mounted so as to be translationally movable on the platform, and a foot support which is provided with a second member for immobilizing the patient's foot on the foot support, such as, for example, arranged as a strap for tying the foot and the base of the leg onto the foot support. The foot support is rotatably mounted on the platform.

The device also comprises a member for positioning the first sensor on the TTA and a second member for positioning the second sensor relative to the foot support.

The device also comprises a system for analyzing the measurements respectively supplied by the first sensor and by the second sensor and relating to their strokes as a result of a translational and rotational movement of the tibia, and an interface for displaying the data supplied by the analysis system from which a diagnosis of at least partial injury to the ACL of the knee is deduced by a practitioner.

According to the invention, the device comprises said first motor assigned to a translational movement of the thrust member and said second motor allocated to a rotational movement of the foot support. The device further comprises said first control module that said control member for correlated operations of the first motor and of the second motor includes, in accordance with the application of said at least one pre-set force model stored in a first directory via a first memory equipping said first control module.

Said at least one force model is assigned to a specific patient and/or is selected by the practitioner, via for example a menu with selectable choices, depending on the examination to be carried out, from among a plurality of pre-set standard force models listed in the first memory. The first directory of force models may vary during the therapeutic monitoring of the patient, by being adapted and/or completed by the practitioner with one or more new force models specific to the patient.

According to one embodiment, at least one said second member for positioning the second sensor is configured to be located on the patient in an area of the patient's leg situated closest to the TTA of the patient's knee.

One said second member for positioning the second sensor is, for example, arranged in said legging that at least partially envelops the patient's calf located near the thrust member, and/or is participating in said articulated beam which is attached to the platform and which is moreover potentially carrying the first sensor.

According to an advantageous embodiment, the first member for positioning the first sensor and said second member for positioning said second sensor are mounted on at least one articulated beam which is attached to the platform.

The potentially alternating or tandem usage of a second sensor mounted on the legging and/or of a second sensor mounted on the beam, allows the practitioner to carry out diversified measurements of the rotation of the tibia. The practitioner can use, selectively or in combination, a second sensor that is mounted on the legging by being located close to the TTA and/or a second sensor mounted on the beam and applied against the TTA as a temporary substitute for the first sensor. Such a combination of using a plurality of second sensors makes it possible to refine the detection of a rotational instability of the knee.

According to one embodiment, the device is provided with an assistance device at least comprising a signal transmitter capable of indicating to a practitioner a correct positioning of the first sensor and/or of the second sensor on a specific patient, with regard to the reference positions respectively of the first sensor and/or of the second sensor on the patient which are previously defined and stored in a second directory of individualized sensor positions for various patients via a second memory of the assistance device.

As a result of the application of the first sensor and/or of the second sensor on the leg of a specific patient during an initial examination, a calibration of the sensors is carried out at reference positions of the sensors which are then stored in the second directory.

During a subsequent examination, especially in the context of therapeutic monitoring of the patient, the practitioner can reproduce the positioning of the first sensor and/or of the second sensor on the same patient in accordance with their respective reference positions previously stored, by being guided by a sound signal and/or by a light signal emitted by the assistance device. Reliable reproducibility of the initial examination is obtained, making more relevant the diagnostics subsequently carried out during the therapeutic monitoring of the patient.

According to one embodiment, the control member includes a second module for controlling at least one configuring of the seat for a specific patient, the second control module being a servo-control module correlated with at least one third motor for positioning in relative tilt between the squab and the backrest, a fourth motor for positioning in relative tilt between the platform and the squab, and preferably a fifth motor for positioning in height the squab.

The second control module comprises a third storage memory of a third directory of individualized seat configuration models for various patients which are pre-set and are reproducible by the control member.

### DESCRIPTION OF THE FIGURES

The invention will be better understood on reading the following detailed description of an example embodiment of the present invention, in relation with the following figure:

FIG. 1 is a schematic profile presentation of a patient installed on a seat that a device according to an example embodiment of the invention comprises.

### DETAILED DESCRIPTION OF THE INVENTION

The figure and its non-limiting detailed description show the invention according to particular conditions which are not restrictive as to the scope of the invention. The figures and their detailed descriptions of an example embodiment of the invention can be used to better define it, if necessary in relation with the general description that has just been given.

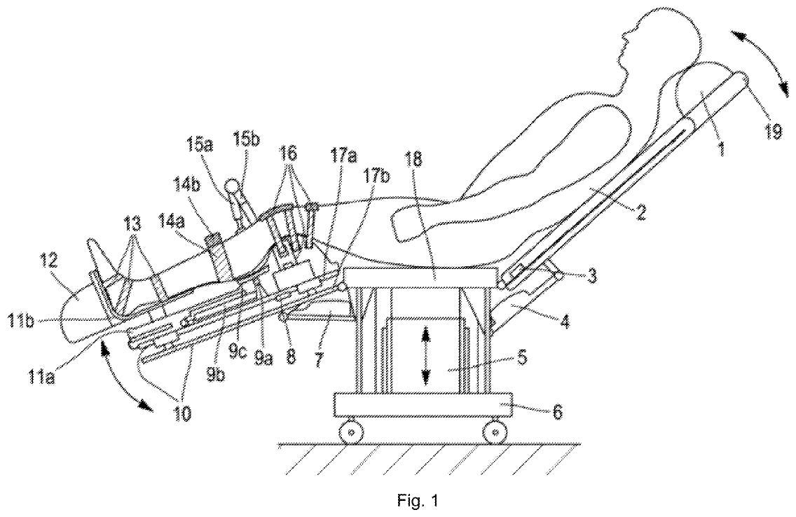

In FIG. 1, a device is organized to evaluate a potential injury to the ACL of a patient's knee. The device comprises a seat (1) for placing the patient in for their examination by a practitioner. The seat (1) includes a squab (18) and a backrest (19) which are tiltable relative to one another, the position of the squab (18) being adjustable in height. A support platform (17a) for supporting a leg of the patient is mounted on a support (17b) which is articulated in pivoting relative to the squab (18)).

The seat (1) can thus be located in various specific configurations for seating the patient on the device, by adjusting the relative positions between the squab (18), the backrest (19) and the platform (17a). During an examination of the patient, the practitioner can thus modify the configuration of the seat (1) according to specific implementations of the device in order to carry out an evaluation at various degrees of bending of the patient's knee.

The platform (17a) is equipped with a base (11a) for supporting the patient's knee. The platform (17a) is also equipped with a foot support (11b) receiving the patient's foot and a thrust member (9a) comprising a shell (9c) on which the patient's calf rests, near his knee.

The patient's knee is firmly held on the base (11a) via a first immobilizing member (16) arranged in a strap tying the knee onto the base (11a). The foot of the patient is firmly held on the foot support (11b) via a second immobilizing member (13) arranged in a strap tying at least the foot and the base of the patient's leg onto the foot support (11b).

The thrust member (9a) is mounted so as to be translationally movable on the platform (17a), in order to exert a thrust force against the patient's calf at a desired force threshold. The foot support (11b) is mounted so as to be able to rotate on the platform (17a), in order to cause the tibia to be rotated at a desired force threshold.

The device is equipped with different motors (4, 5, 7) to adjust the configuration of the seat (1) in various stations of examination of the patient. The motor (4) makes it possible to adjust the tilt between the squab (18) and the backrest (19). The motor (7) makes it possible to adjust the tilt between the squab (18) and the platform (17a) via the support 10 carrying the platform (17a). The motor (5) makes it possible to adjust the height position of the squab (18).

The device is also equipped with different motors (9b, 12) to place the patient's tibia under stress in translation and/or in rotation. A first motor (9b), such as especially an electric cylinder, is assigned to the translational operation of the thrust member (9a), in order to cause a translational movement of the tibia relative to the femur. A second motor (12), such as especially an electric rotary motor, is assigned to the rotational operation of the foot support (11b), in order to cause the patient's tibia to be rotated.

The device is also equipped with a first sensor (15a) for measuring the translational movement of the tibia as a result of the activation of the first motor (9b) causing the translational movement of the thrust member (9a).

The first sensor (15a), such as a proximity sensor with potential difference, is mounted on a beam (15b) attached to the platform (17a) by being applied against the TTA during the examination of the patient. A second sensor (14b) such as a proximity sensor with potential difference configured as an inclinometer and/or accelerometer is mounted in the example shown on a legging (14a) enveloping the patient's calf by being applied against the tibia near the first sensor (15), as close as possible to the TTA.

A human/machine interface, hereinafter referred to as a HMI, allows the practitioner to establish their diagnosis from logical processing by a system for analyzing measurements supplied at least by the first sensor (15a) and the second sensor (14b) which are carried out at different degrees of bending of the knee by tilting the platform (17a) with respect to the squab (18) according to the predefined configurations of the seat (19).

At the end of a sequence of examination of the patient, an interface for displaying the logical processing of the measurements performed by the analysis system, provides a display of the obtained result in the form of a diagram. The diagram includes two comparative curves, one a reference curve relating to an absence of injury to the ACL (green curve) and the other a curve relating to a possible injury to the ACL (red curve) identified by the analysis system on the basis of the processing of the measurements carried out.

To perform the examination of the patient, a control member included in the HMI comprises a second control module which regulates the operation of motor (4), motor (5) and motor (7) to place the seat (19) in configurations previously defined with different degrees of knee bending.

The configurations of the seat (19) are then applied according to configuration models of the seat (19) which are selectable and/or adaptable by the practitioner, and which are classified according to a third directory previously established and stored in a third memory of the second control module.

Prior to an examination of the patient, the first sensor (15a) and the second sensor (14b) are calibrated by being located at reference positions (15b, 14a), respectively against the TTA for the first sensor (15a) and in proximity to the TTA for the second sensor (14b) according to the shown example. The HMI comprises an assistance device guiding the practitioner to reproduce the reference positions (15b), (14a) of the first sensor (15a) and of the second sensor (14b) to perform an examination of the patient. The respective reference positions (15b, 14a) of the sensors (15a, 14b) are classified according to a second directory previously established and stored in a second memory of the assistance device.

Subsequently, during therapeutic monitoring of the patient, the installation positions of the sensors (15b, 14a) by the practitioner are compared with their respective reference positions (15a, 14b), in order to define the relevance of subsequent positioning of the sensors (15a, 14b) on the patient. In the event of a position shift of the sensors (15a and 14b) relative to their previously stored reference positions (15b, 14a), the practitioner is then guided via an emitter of a sound and/or light signal to place the first sensor (15a) and the second sensor (14b) on the patient in accordance with their respective reference positions (15a, 14b).

To perform an examination of the patient, the practitioner uses the HMI to cause the translational movement of the thrust member (9a) and the rotation of the foot support (11b). For this purpose, the control member comprises a first control module for implementing the first motor (9b) and the second motor (12), which are controlled in correlation by the first control module.

More particularly for an examination phase of the patient, the forces and/or the amplitudes of stressing of the tibia translationally—via the first motor (9b)—and rotationally—via the second motor (12) are controlled according to force models selectable and/or adaptable by the practitioner, which are classified based on a first directory, pre-set and stored in a first memory. Each of the force models comprises a first force law regulating the operation of the first motor (9b) and a second force law regulating the operation of the second motor (12).

The first motor (9b) and the second motor (12) are thus controlled in combination, by applying a force model—combining in correlation said first force law and said second force law—which is selected by the practitioner among various force models listed in the first memory, and this for various degrees of bending of the knee.

The operation of the first motor 9a and of the second motor 12 according to a force model selected by the practitioner makes it possible to regulate—via the control member—conditions for stressing the tibia as desired by the practitioner. On the basis of the application of a force model, a combined servo-control of the first motor (9a) and of the second motor (12) by the first control module makes it possible to subject the patient's tibia to a translational and rotational stress according to forces and amplitudes which are correlated, with the ability to be selectively applied—per the practitioners demands—simultaneously or consecutively.

The various measurements of the translational and/or rotational movement of the tibia are selectively carried out by the practitioner according to pre-defined force models and adapted to a specific patient; from said force models, the respective operations of the first motor (9b) and of the second motor (12) are correlated. The force models are pre-set on the basis of standardized force models or on the basis of specific force models constructed and/or potentially adapted during an initial examination of a patient, by being reproducible during therapeutic monitoring of the patient and/or adapted according to the progress of the injury to the ACL identified during the initial examination.

A precise and reliable diagnosis may thus be rapidly made by the practitioner, taking into account a rotational instability of the tibia evaluated accurately on the basis of various measurements of a translation of the tibia and of a rotation of the tibia which are correlated by the combined control of the operations of the first motor (9b) according to said first specific force law and of the second motor (12) according to said second specific force law.

The reproducibility of the examination during therapeutic monitoring is made reliable and/or can be adapted depending on the progress of the injury to the patient's ACL, from a choice by the practitioner of the force models to be applied and/or adapted. The accuracy of the measurements carried out is also reinforced by applying the first sensor (15a) and/or the second sensor (14b) against the TTA, or at least as close as possible to the TTA.

The analysis system can provide performance and comparative results between measurements whose measurement conditions can be diversified by being easy to implement by the practitioner on the basis of pre-set and/or adapted force models. The mutual servo-control of the stresses applied to the tibia for its translational movement for various configurations of the seat—under the control of the operation of the first motor (9b) and of the second motor (12) by the control member—contribute to the quality and accuracy obtained—via the analysis system—from the analysis of the measurements carried out.

### USE OF THE PATENTED EQUIPMENT

The aforementioned device is especially intended for preoperative diagnosis.

It makes it possible to implement a method for measuring a rotational movement of a patient's knee in combination with a measurement of a translational movement of the associated tibia relative to the associated femur of the patient at different degrees of bending of the knee, the method being implemented by a device at least comprising a seat (1) for placing the patient in, including a squab (18) extended by an inclinable back rest (19) relative to the squab (18), and including a platform (17a) for supporting a leg of the patient which is pivotably mounted in position on the squab (18), the platform (17a) being equipped with a first member (16) for immobilizing the patient's knee on the platform (17a), a foot support (11b) which is provided with a second member for immobilizing (13) the patient's foot and which is mounted so as to be rotationally movable on the platform (10), and a thrust member (9a) for pushing the patient's knee which is mounted so as to be translationally movable on the platform (17a), the device being equipped with a first sensor (15a) for measuring a translational movement of the tibia relative to the femur located on the TTA, and a second sensor (14b) for measuring an angle of rotation of said tibia, close to the TTA, wherein the method of collecting and logical processing is performed by a measurement analysis system at least comprising a first measurement provided by the first sensor (15a) and a second measurement provided by the second sensor (14b) for each of said different degrees of bending of the knee, characterized in that:
the measurement of the translational movement of the tibia is carried out by the first measurement sensor (15a) from a translation of the thrust member (9a) on the platform (17a) which is performed by a first motor (9b),
the measurement of the angle of rotation of the tibia is carried out by the second sensor (14b) located as dose as possible to the TTA of the patient's knee, from a rotation of the foot support (11b) which is performed by a second motor (12), and
The respective operations of the first motor (9b) and of the second motor (12) are correlated by a first control module that a control member includes,
the respective activation conditions of the first motor (9b) and of the second motor (12) are mutually controlled in at least one listed force model for stressing the tibia of a specific patient that is pre-set and reproducible and/or adaptable to the patient in question,
said at least one force model combines, for each of the degrees of bending of the patient's knee, on the one hand a first force law relating to the translational movement of the tibia, via the thrust member (9a) moved in translation on the platform (17a) by the first motor (9b), and on the other hand a second force law relating to the rotation of the patient's foot, via the foot support (11b), by the second motor (12) for rotating the foot support (11b).

Preferably, the second sensor (14b) is located near the thrust member (9a) following the extension of the leg, the first sensor (15a) being located on the TTA and the second sensor (14b) being located adjacent to the first sensor (15a), close to the TTA.

Advantageously, the second sensor (14b) is mounted on a legging (14a) installed around the patient's calf.

According to first conditions of examination, the measurement of the translational movement of the tibia by the first sensor (15a) and the measurement of the angle of rotation of the tibia by the second sensor (14b), are carried out simultaneously under the control of the control member in accordance with said at least one force model.

According to second conditions of examination, the measurement of the translational movement of the tibia by the first sensor (15a) and the measurement of the angle of rotation of the tibia by the second sensor (14b), are carried out consecutively under the control of the control member in accordance with said at least one force model.

Advantageously, prior to a reading of measurements on the patient, the first sensor (15a) and the second sensor (14b) are calibrated at respective reference positions (15b, 14a) by examining a healthy leg of the patient subjected to said at least one force model.

Preferably, the control member includes a second servo-control module correlated with at least one third motor (4) for positioning in relative tilt between the squab (18) and the backrest (19), a fourth motor (7) for positioning in relative tilt between the platform (17b) and the squab (18), and a fifth motor (5) for positioning in height the squab (18), the respective strokes of the third motor (4), the fourth motor (7) and the fifth motor (5) being mutually controlled according to at least one pre-set and reproducible configuration model of the seat (3 and 8) for a specific patient.

1. A measuring device for characterizing an anterior cruciate ligament injury to a patient's knee of a patient, comprising a seat provided with a squab having a tiltable backrest, a support platform for supporting the patient's leg, rotationally movable relative to the squab, and provided with a thrust member for pushing the patient's calf equipped with a first member for immobilizing the patient's knee on the platform and with a thrust member for pushing the knee mounted translationally movable on the platform wherein said device further comprises:
a foot support, rotationally movable on the platform, capable of causing the tibia to rotate at a desired force threshold, said foot support being provided with a second immobilization member for immobilizing the patient's foot;
a first sensor capable of being positioned on the TTA of said patient as well as a second sensor assigned to measuring an angle of rotation of the tibia and mounted on a member for positioning said second sensor relative to the foot support; and
a system for analyzing measurements respectively supplied by the first sensor and by the second sensor and relating to their strokes as a result of a respective translational and rotational movement of the tibia, and an interface for displaying the data supplied by the analysis system.

2. The measuring device for characterizing an injury to the anterior cruciate ligament of a patient's knee according to claim 1, wherein the device comprises a first motor controlling the translation of the thrust member and a second motor controlling a rotation of the foot support.

3. The measuring device for characterizing an injury to the anterior cruciate ligament of a patient's knee according to claim 1, wherein the device comprises a computer memory for recording at least one digital force model and a computer controlling said motors by applying control laws depending on the contents of said computer memory.

4. The measuring device for characterizing an injury to the anterior cruciate ligament of a patient's knee according to claim 1, wherein the second member for positioning said second sensor is arranged in said legging.

5. The measuring device for characterizing an injury to the anterior cruciate ligament of a patient's knee according to claim 1, wherein the first member for positioning the first sensor and said second member for positioning said second sensor are mounted on at least one articulated beam which is attached to the platform.

6. The measuring device for characterizing an injury to the anterior cruciate ligament of a patient's knee according to claim 1, wherein it is also provided with an assistance device at least comprising a signal transmitter capable of indicating to a practitioner correct positioning of the first sensor and/or of the second sensor on a specific patient, with regard to reference positions respectively of the first sensor and/or of the second sensor on the patient previously defined and stored in a second directory of individualized sensor positions for various patients via a second memory of the assistance device.

7. The device according to claim 1, wherein the control member includes a second module for controlling at least one configuring of the seat for a specific patient, the second control module being a servo-control module correlated with at least one third motor for positioning in relative inclination between the squab and the backrest, a fourth motor for positioning in relative inclination between the platform and the squab, the second control module comprising a third memory for storing a third directory of individualized seat configuration models for various patients which are pre-set and are reproducible by the control member.

8. The measuring device for characterizing an injury to the anterior cruciate ligament of a patient's knee according to claim 2, wherein the device comprises a computer memory for recording at least one digital force model and a computer controlling said motors by applying control laws depending on the contents of said computer memory.

9. The measuring device for characterizing an injury to the anterior cruciate ligament of a patient's knee according to claim 2, wherein the second member for positioning said second sensor is arranged in said legging.

10. The measuring device for characterizing an injury to the anterior cruciate ligament of a patient's knee according to claim 2, wherein the first member for positioning the first sensor and said second member for positioning said second sensor are mounted on at least one articulated beam which is attached to the platform.

11. The measuring device for characterizing an injury to the anterior cruciate ligament of a patient's knee according to claim 2, wherein it is also provided with an assistance device at least comprising a signal transmitter capable of indicating to a practitioner correct positioning of the first sensor and/or of the second sensor on a specific patient, with regard to reference positions respectively of the first sensor and/or of the second sensor on the patient previously defined and stored in a second directory of individualized sensor positions for various patients via a second memory of the assistance device.

12. The device according to claim 2, wherein the control member includes a second module for controlling at least one configuring of the seat for a specific patient, the second control module being a servo-control module correlated with at least one third motor for positioning in relative inclination between the squab and the backrest, a fourth motor for positioning in relative inclination between the platform and the squab, the second control module comprising a third memory for storing a third directory of individualized seat configuration models for various patients which are pre-set and are reproducible by the control member.
